(19) United States
(12) Patent Application Publication
(10) Pub. No.: US 2025/0360125 A1
(43) Pub. Date: Nov. 27, 2025

(54) METHODS AND COMPOSITIONS FOR TREATING HUNTINGTON'S DISEASE AND ITS SYMPTOMS

(71) Applicant: Corcept Therapeutics Incorporated, Menlo Park, CA (US)

(72) Inventors: Hazel Hunt, Storrington (GB); Joseph Belanoff, Woodside, CA (US); Onno C. Meijer, Leiden (NL); Max Gentenaar, Leiden (NL); Willeke M.C. van Roon-Mom, Leiden (NL)

(73) Assignee: Corcept Therapeutics Incorporated, Menlo Park, CA (US)

(21) Appl. No.: 18/686,112

(22) PCT Filed: Feb. 16, 2024
(86) PCT No.: PCT/US2024/016080

§ 371 Date: Feb. 23, 2024

Related U.S. Application Data
(60) Provisional application No. 63/446,711, filed on Feb. 17, 2023.

(51) Int. Cl. A61K 31/4745; A61P 25/08; A61P 25/14

(52) U.S. Cl. CPC A61K 31/4745; A61P 25/08; A61P 25/14

(57) ABSTRACT

Methods and compositions for treating a patient suffering from Huntington's Disease or for treating symptoms of Huntington's Disease are disclosed. The methods include administration of an effective amount of a heteroaryl ketone fused azadecalin glucocorticoid receptor modulator (GRM) or an octahydro fused azadecalin GRM to the patient. In embodiments, the GRM is dazucorilant. In embodiments, the GRM is zavacorilant. In embodiments, the GRM is orally administered. In some embodiments, the GRM is administered daily; in other embodiments, the GRM is administered on a schedule such as, e.g., once every other day, once every three days, once per week, or other administration schedule. Symptoms of Huntington's Disease that may be treated by the present methods include, without limitation, motor symptoms (e.g., muscle weakness, postural irregularities, difficulty walking, difficulty swallowing), and neurological or psychological symptoms (e.g., epileptic seizures, amnesia; confusion; impaired speech; delirium; depression; anxiety).

### BACKGROUND

Huntington's Disease (HD), also known as Huntington's Chorea, is a genetic neurodegenerative disease in which patients show impaired motor functions and cognitive decline, psychiatric disorders, muscle wasting and metabolic dysfunctions. As defined by the World Federation of Neurology, chorea is characterized by excessive, spontaneous movements, that are irregularly timed, non-repetitive, randomly distributed and abrupt in character. Such movements are termed “choreic” movements.

HD is caused by an excessive number of CAG repeats (35+) in the huntingtin gene. This increase of CAG repeats results in the mutated huntingtin protein (mHtt), which gains a toxic function and partially loses its natural function. This mutated protein mHtt promotes pathological interactions and aggregates in the brain leading to cellular malfunction and eventually neuronal death. The mHtt aggregates are the neuropathological hallmarks of the disease. Patients suffering from HD experience progressive worsening of cognitive, motor, and metabolic dysfunctions. HD is a terminal disease, with death typically occurring within about 15 to 30 years from initial symptom onset.

Current treatments for HD are mainly symptomatic, limited to treatments for helping the patient cope with the disease, and to cope with the psychological aspects of living with a progressive and terminal neurological disease. Tetrabenazine may be prescribed in an attempt to reduce the frequency or severity of sudden or unusual choreic limb movements associated with HD. Although not approved for such use, drugs such as haloperidol, risperidone, chlorpromazine, and amantadine may also be used in an attempt to reduce the frequency or severity of such unwanted choreic movements associated with HD. Antidepressants or antianxiety drugs may be prescribed in an attempt to reduce the depression or anxiety experienced by many sufferers of HD. High levels of endogenous glucocorticoids have been linked to several HD-related symptoms including neurodegeneration, cognitive decline, muscular atrophy and metabolic dysfunctions¹⁻³. The R6/2 mouse strain, the most commonly used HD model, exhibits increased glucocorticoid levels, mHtt aggregates, and various motor symptoms⁴⁻⁶.

The glucocorticoids cortisol (in, e.g., humans) and corticosterone (in e.g., rodents) are steroid hormones produced in the adrenal glands with widespread actions throughout the body. Cortisol and corticosterone act via binding to a glucocorticoid receptor (GR). The most commonly used GR modulator (GRM), mifepristone (RU486), is not specific for GR; it also has affinity for other nuclear steroid receptors such as, e.g., the progesterone receptor (PR) and the androgen receptor (AR). This lack of selectivity for GR can contribute to side effects related to PR or AR activity when administered for use directed to GR, or to side effects related to GR activity when administered for use directed at, for example, PR or AR.

Accordingly, there is need in the art for additional and effective treatments for HD. Where such treatments relate to cortisol activity in humans, GRMs more selective for GR than mifepristone would be desired.

### SUMMARY

Disclosed herein are novel methods for treating Huntington's Disease (HD) and for alleviating symptoms related to HD. The methods comprise administering to the subject an effective amount of a glucocorticoid receptor modulator (GRM) effective to treat a patient suffering from HD, and effective to treat patient symptoms that are related to HD. Such symptoms may include, without limitation, motor symptoms, neurological symptoms, and psychological symptoms.

Motor symptoms may include, for example and without limitation, involuntary jerking motions (spasms); involuntary writhing motions (chorea); muscular contractions or rigidity (dystonia); tremor; slowed or unusual eye movements; impaired muscle strength; impaired grasp; impaired gait (i.e., difficulty walking); impaired balance; impaired swallowing; impaired respiration; impaired posture; impaired ability to stand upright; impaired ability to maintain head position; impaired speech; and other motor symptoms, where impairment is determined with comparison to baseline ability to perform the motor activity (e.g., before onset of HD symptoms, or upon initial diagnosis of HD symptoms).

Neurological and psychological symptoms may include, for example and without limitation, epileptic seizures; amnesia; other memory loss; mental confusion; impaired speech; impaired ability to concentrate; impaired speed of comprehension; delirium; hallucinations; paranoia; depression; anxiety; apathy; rapid or unprovoked changes in mood; and other neurological or psychological symptoms, where impairment is determined with comparison to baseline ability or level of the neurological or psychological activity or symptom (e.g., before onset of HD symptoms, or upon initial diagnosis of HD symptoms).

In embodiments, the GRM is a selective GRM (SGRM) active at GR but with little or no activity at other steroid hormone receptors (e.g., little or no PR or AR activity). In embodiments, the GRM is a nonsteroidal compound comprising a heteroaryl ketone fused azadecalin structure, wherein the heteroaryl ketone fused azadecalin structure is as described and disclosed in U.S. Pat. No. 8,859,774. In embodiments, the GRM is a nonsteroidal compound comprising an octahydro fused azadecalin structure, wherein the octahydro fused azadecalin structure is as described and disclosed in U.S. Pat. No. 10,047,082.

In embodiments, the GRM is the compound comprising a heteroaryl ketone fused azadecalin structure (R)-(1-(4-fluorophenyl)-6-((4-(trifluoromethyl)phenyl) sulfonyl)-4, 4a, 5,6,7,8-hexahydro-1-H-pyrazolo P,4-g]isoquinolin-4a-yl) (pyridin-2-yl)methanone (termed “dazucorilant” or “CORT113176”), which has the following structure:

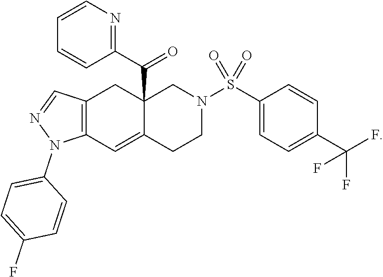 [SMILES: O=C(c1ccccn1)[C@]12Cc3cnn(-c4ccc(F)cc4)c3C=C1CCN(S(=O)(=O)c1ccc(C(F)(F)F)cc1)C2]

In embodiments, the GRM is the compound comprising an octahydro fused azadecalin structure ((4aR,8aS)-1-(4-fluorophenyl)-6-((2-isopropyl-2H-1,2,3-triazol-4-yl)sulfonyl)-4,4a,5,6,7,8,8a,9-octahydro-1H-pyrazolo[3,4-g]isoquinolin-4a-yl)(thiazol-4-yl)methanone (termed zavacorilant, or “CORT125329”), which has the following structure:

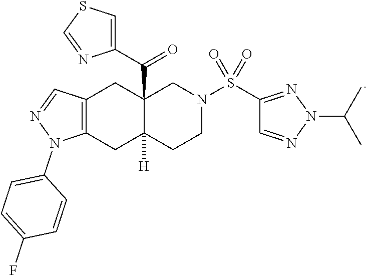 [SMILES: CC(C)n1ncc(S(=O)(=O)N2CC[C@H]3Cc4c(cnn4-c4ccc(F)cc4)C[C@]3(C(=O)c3cscn3)C2)n1]

The entire contents of U.S. Pat. No. 8,859,774 and of U.S. Pat. No. 10,047,082 are hereby incorporated by reference in their entireties.

In embodiments, the GRM is administered orally to the patient. GRM treatment for HD, or for HD symptoms, may include daily administration of the GRM (e.g., once per day, or twice per day, or other daily schedule of administration); or may include intermittent GRM administration (e.g., once every other day, or once every three days, or twice per week, or other schedule of administration). In embodiments, the effective amount of the GRM is a dose of between about 1 and about 100 milligrams per kilogram (mg/kg). For example, a GRM dose for daily GRM administration to treat HD, or to treat symptoms of HD, is a dose of between about 1 and about 100 milligrams per kilogram per day (mg/kg/day), or between about 3 mg/kg/day to about 75 mg/kg/day, or between about 5 mg/kg/day to about 50 mg/kg/day. In embodiments, the daily dose of the GRM is between about 5 milligrams per day (mg/day) and about 3000 mg/day, or between about 10 mg/day and about 2500 mg/day, or between about 20 mg/day and about 2250 mg/day, or between about 30 mg/day and about 2000 mg/day, or between about 40 mg/day and about 1750 mg/day, about 50 mg/day and about 1500 mg/day, or between about 75 mg/day and about 1000 mg/day, or between about 100 mg/day to about 750 mg/day, or between about 150 mg/day to about 500 mg/day. GRM treatment may be administered for as long as needed; thus, such treatment may be for a period of one year, or for a period of several years, or may be administered many years. In embodiments, GRM treatment comprises GRM administration for at least 1 to about 80 weeks, or more. The GRM may be administered along with other medications or treatments administered to the patient suffering from HD, or during a period of time in which the patient suffering from HD is receiving other medication or treatment for HD or its symptoms.

The present methods provide improved methods of treating HD and for treating symptoms of HD.

### BRIEF DESCRIPTION OF THE DRAWINGS

FIG. 1A. Effects of the HD genotype and CORT113176 treatment on functional parameters in mice. Forelimb grip strength measured over time of male mice. Data presented as mean+S.E.M. and analysed by three-way ANOVA or mixed effect model. Individual timepoints were analysed by two-way ANOVA with Tukey's multiple comparison represented as *p<0.05, +=WT-Veh vs HD-CORT113176.

FIG. 1B. Effects of the HD genotype and CORT113176 treatment on functional parameters in mice. Hindlimb clasping score measured over time of male mice. Data presented as mean+S.E.M. and analysed by three-way ANOVA or mixed effect model. Individual timepoints were analysed by two-way ANOVA with Tukey's multiple comparison represented as ***p<0.001,****p<0.0001 (*=WT-Veh vs HD-Veh, $=WT-CORT113176 vs HD-Veh, #=HD-CORT113176 vs HD-Veh).

FIG. 1C. Effects of the HD genotype and CORT113176 treatment on functional parameters in mice. Hindlimb clasping score measured over time of female mice. Data presented as mean+S.E.M. and analysed by three-way ANOVA or mixed effect model. Individual timepoints were analysed by two-way ANOVA with Tukey's multiple comparison represented as *p<0.05, **p<0.01, ****p<0.0001 (*=WT-Veh vs HD-Veh, $=WT-CORT113176 vs HD-Veh, +=WT-Veh vs HD-CORT113176 and Ω=WT-CORT113176 vs HD-CORT1113176).

FIG. 1D. Effects of the HD genotype and CORT113176 treatment on functional parameters in mice. Total number of epileptic seizures of both male and female mice.

FIG. 1E. Effects of the HD genotype and CORT113176 treatment on functional parameters in mice. Kaplan-Meier analysis for first occurrences of epilepsy in male mice.

FIG. 1F. Effects of the HD genotype and CORT113176 treatment on functional parameters in mice. Kaplan-Meier analysis for first occurrences of epilepsy in female mice. Epileptic seizures occurred most frequently in the vehicle-treated female HD mice, while none were observed in the CORT113176-treated female HD mice.

FIG. 2A. Immunohistochemistry of markers related to the HD genotype in the striatum of male mice. The effect of the HD genotype and CORT113176 on GFAP intensity in the male striatum. Data are presented as mean+S.E.M., and were analysed by two-way ANOVA with Tukey's multiple comparison represented as * p<0.05, **p<0.01, and ***p<0.001.

FIG. 2B. Immunohistochemistry of markers related to the HD genotype in the striatum of female mice. The effect of the HD genotype and CORT113176 on GFAP intensity in the female striatum. Data are presented as mean+S.E.M. and were analysed by two-way ANOVA with Tukey's multiple comparison represented as * p<0.05, and **p<0.01.

FIG. 2C. Immunohistochemistry of markers related to the HD genotype in the hippocampus of male mice. The effect of the HD genotype and CORT113176 on GFAP intensity in the male hippocampus. Data are presented as mean+S.E.M. and were analysed by two-way ANOVA with Tukey's multiple comparison represented as * p<0.05.

FIG. 2D. Immunohistochemistry of markers related to the HD genotype in the hippocampus of female mice. The effect of the HD genotype and CORT113176 on GFAP intensity in the female hippocampus. Data are presented as mean+S.E.M.

FIG. 2E. Immunohistochemistry of markers related to the HD genotype in the striatum and hippocampus of male mice. The effect of the HD genotype and CORT113176 on Iba1 intensity in the male hippocampus. Data are presented as mean+S.E.M.

FIG. 3A. The effect of the HD genotype and CORT113176 on mHtt aggregates in the striatum in male R6/2 mice. Average mHtt aggregate size within the striatum of male R6/2 mice. Data presented as mean+S.E.M. and were analysed by one-way ANOVA with Tukey's multiple comparison represented as ****p<0.0001.

FIG. 3B. The effect of the HD genotype and CORT113176 on mHtt aggregates in the striatum in male R6/2 mice. Total mHtt area within the striatum of male R6/2 mice. Data presented as mean+S.E.M. and were analysed by one-way ANOVA with Tukey's multiple comparison represented as * p<0.05, and **p<0.01.

### DETAILED DESCRIPTION

The methods disclosed herein can be used to treat a patient suffering from Huntington's Disease (HD) by administering an effective amount of a heteroaryl ketone fused azadecalin glucocorticoid receptor modulator (GRM), or an octahydro fused azadecalin GRM, effective to treat HD. In embodiments, the heteroaryl ketone fused azadecalin GRM is the compound (R)-(1-(4-fluorophenyl)-6-((4-(trifluoromethyl)phenyl) sulfonyl)-4, 4a, 5,6,7,8-hexahydro-1-H-pyrazolo P,4-g]isoquinolin-4a-yl) (pyridin-2-yl)methanone (termed “dazucorilant” or “CORT113176”), which has the following structure:

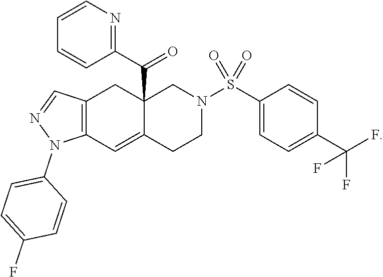 [SMILES: O=C(c1ccccn1)[C@]12Cc3cnn(-c4ccc(F)cc4)c3C=C1CCN(S(=O)(=O)c1ccc(C(F)(F)F)cc1)C2]

The GRM CORT113176 lacks significant cross-reactivity with other steroid receptors. In embodiments, the octahydro fused azadecalin GRM is the compound ((4aR,8aS)-1-(4-fluorophenyl)-6-((2-isopropyl-2H-1,2,3-triazol-4-yl)sulfonyl)-4,4a,5,6,7,8,8a,9-octahydro-1H-pyrazolo[3,4-g]isoquinolin-4a-yl)(thiazol-4-yl)methanone (termed zavacorilant, or “CORT125329”), which has the following structure:

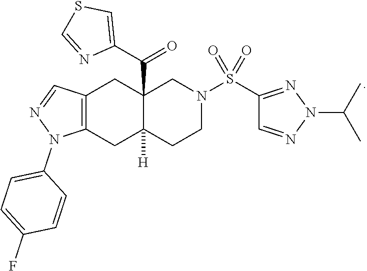 [SMILES: CC(C)n1ncc(S(=O)(=O)N2CC[C@H]3Cc4c(cnn4-c4ccc(F)cc4)C[C@]3(C(=O)c3cscn3)C2)n1]

The GRM zavacorilant lacks significant cross-reactivity with other steroid receptors.

Accordingly, Applicant discloses herein methods of treating HD, and symptoms of HD, comprising administering an effective amount of a GRM to a patient suffering from HD. In embodiments, the GRM is a heteroaryl ketone fused azadecalin GRM; in a particular embodiment, the GRM is dazucorilant. Accordingly, uses of heteroaryl ketone fused azadecalin GRMs (including, e.g., dazucorilant) for treating HD and symptoms thereof are disclosed herein. Applicant also discloses herein the use of a heteroaryl ketone fused azadecalin GRM (e.g., dazucorilant) in the manufacture of a medicament for treating HD and for treating symptoms of HD.

Applicant further discloses herein pharmaceutical compositions comprising a heteroaryl ketone fused azadecalin GRM for treating HD. In embodiments, the heteroaryl ketone fused azadecalin GRM is dazucorilant. Such pharmaceutical compositions include, for example, capsules, tablets, pills, solutions, and emulsions comprising a heteroaryl ketone fused azadecalin GRM (e.g., dazucorilant).

Accordingly, Applicant discloses herein that HD and symptoms of HD may be treated by administration of an effective amount of a heteroaryl ketone fused azadecalin GRM, such as, e.g., dazucorilant. Symptoms of HD that may be treated by administration of an effective amount of a heteroaryl ketone fused azadecalin GRM, such as, e.g., dazucorilant, including, without limitation, motor symptoms, neurological symptoms, and psychological symptoms.

As discussed above, Huntington's Disease (HD) is a genetic neurodegenerative disease caused by a mutation in the huntingtin gene. Mutant huntingtin (mHtt) leads to cellular malfunction, protein aggregates and eventually results in neuronal cell death. Patients suffering from HD show impaired motor functions, neurological symptoms and impairment, cognitive decline, and may have epileptic seizures. Elevated levels of glucocorticoids have been found in patients with HD and in HD mouse models. Applicant discloses herein the results of studies that evaluated the efficacy of the selective GRM CORT113176 in the commonly used R6/2 mouse model. This mouse model is characterized by severe motor decline in the course of weeks. In male mice, CORT113176 treatment significantly delayed the loss of grip strength, the development of hindlimb clasping, gait abnormalities, and the occurrence of epileptic seizures. CORT113176 treatment also reduced clasping behavior and the occurrence of epileptic seizures in female mice. CORT113176 administration restored parameters that were changed in HD including astrocyte markers in both striatum and hippocampus as well as microglia markers in hippocampus. CORT113176 delayed the formation of mHtt aggregates in the striatum and the hippocampus. Applicant discloses results herein that demonstrate that the heteroaryl ketone fused azadecalin GRM CORT113176 can effectively delay several key symptoms related to the HD phenotype in mice. Accordingly, Applicant discloses herein that heteroaryl ketone fused azadecalin GRMs such as CORT113176 administered to patients suffering from HD or from symptoms of HD is an effective treatment for HD and its symptoms.

### Definitions

Citations to scientific references are indicated by superscript numbers that refer to the list of References included at the end of this specification.

As used herein, the term “patient” refers to a human that is or will be receiving, or has received, medical care for a disease or condition.

As used herein, the terms “administer,” “administering,” “administered” or “administration” refer to providing a compound or a composition (e.g., one described herein), to a subject or patient. For example, a compound or composition may be administered orally to a patient.

As used herein, the term “effective amount” or “therapeutic amount” refers to an amount of a pharmacological agent effective to treat, eliminate, or mitigate at least one symptom of the disease being treated. In some cases, “therapeutically effective amount” or “effective amount” can refer to an amount of a functional agent or of a pharmaceutical composition useful for exhibiting a detectable therapeutic or inhibitory effect. The effect can be detected by any assay method known in the art. The effective amount can be an amount effective to invoke a therapeutic response.

As used herein, the terms “administer,” “administering,” “administered” or “administration” refer to providing a compound or a composition (e.g., one described herein), to a subject or patient. Administration may be by oral administration (i.e., the subject receives the compound or composition via the mouth, as a pill, capsule, liquid, or in other form suitable for administration via the mouth. Oral administration may be buccal (where the compound or composition is held in the mouth, e.g., under the tongue, and absorbed there). Administration may be by injection, i.e., delivery of the compound or composition via a needle, microneedle, pressure injector, or other means of puncturing the skin or forcefully passing the compound or composition through the skin of the subject. Injection may be intravenous (i.e., into a vein); intraarterial (i.e., into an artery); intraperitoneal (i.e., into the peritoneum); intramuscular (i.e., into a muscle); or by other route of injection. Routes of administration may also include rectal, vaginal, transdermal, via the lungs (e.g., by inhalation), subcutaneous (e.g., by absorption into the skin from an implant containing the compound or composition), or by other route.

As used herein, the term “combination therapy” refers to the administration of at least two pharmaceutical agents to a subject to treat a disease. The two agents may be administered simultaneously, or sequentially in any order during the entire or portions of the treatment period. The at least two agents may be administered following the same or different dosing regimens. In some cases, one agent is administered following a scheduled regimen while the other agent is administered intermittently. In some cases, both agents are administered intermittently. In some embodiments, the one pharmaceutical agent, e.g., a SGRM, is administered daily, and the other pharmaceutical agent, e.g., a other agent, is administered every two, three, or four days.

As used herein, the term “compound” is used to denote a molecular moiety of unique, identifiable chemical structure. A molecular moiety (“compound”) may exist in a free species form, in which it is not associated with other molecules. A compound may also exist as part of a larger aggregate, in which it is associated with other molecule(s), but nevertheless retains its chemical identity. A solvate, in which the molecular moiety of defined chemical structure (“compound”) is associated with a molecule(s) of a solvent, is an example of such an associated form. A hydrate is a solvate in which the associated solvent is water. The recitation of a “compound” refers to the molecular moiety itself (of the recited structure), regardless of whether it exists in a free form or an associated form.

As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The term “glucocorticosteroid” (“GC”) or “glucocorticoid” refers to a steroid hormone that binds to a glucocorticoid receptor. Glucocorticosteroids are typically characterized by having 21 carbon atoms, an α,β-unsaturated ketone in ring A, and an α-ketol group attached to ring D. They differ in the extent of oxygenation or hydroxylation at C-11, C-17, and C-19; see Rawn, “Biosynthesis and Transport of Membrane Lipids and Formation of Cholesterol Derivatives,” in Biochemistry, Daisy et al. (eds.), 1989, pg. 567.

As used herein, the term “glucocorticoid receptor” (“GR”) refers to the type II GR, a family of intracellular receptors which specifically bind to cortisol and/or cortisol analogs such as dexamethasone (See, e.g., Turner & Muller, J. Mol. Endocrinol. Oct. 1, 2005 35 283-292). The glucocorticoid receptor is also referred to as the cortisol receptor. The term includes isoforms of GR, recombinant GR and mutated GR.

The term “cortisol” refers to the naturally occurring glucocorticoid hormone (also known as hydrocortisone) that is produced by the zona fasciculata of the adrenal gland. Cortisol is the glucocorticoid hormone active in humans. Cortisol has the structure:

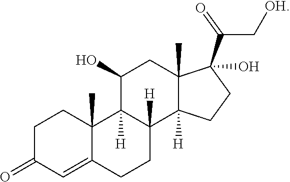

Cortisol may be measured from blood samples, saliva samples, urine samples, and from samples of other bodily fluids. Blood levels (e.g. serum cortisol levels) are believed to reflect short-term cortisol levels, and multiple blood samples from a single subject may show changes in cortisol levels measured over a period of hours. Urinary free cortisol (UFC) and salivary cortisol measurements are believed to reflect daily or longer-term cortisol levels, and so may be useful for overall cortisol measurements summed over circadian variations during the day. The term “total cortisol” refers to cortisol that is bound to cortisol-binding globulin (CBG or transcortin) in the blood and free cortisol (cortisol that is not bound to CBG). The term “free cortisol” refers to cortisol that is not bound to cortisol-binding globulin (CBG or transcortin) in the blood. As used herein, the term “cortisol” refers to total cortisol, free cortisol, and/or cortisol bound of CBG.

The term “corticosterone” refers to the naturally occurring glucocorticoid hormone produced in adrenal glands that is active in rodents such as mice and rats. Corticosterone has the structure:

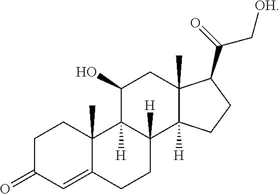

The term “normal level” refers to the average level of an analyte as determined by measurements of samples obtained from multiple normal subjects.

The terms “normal cortisol level” and “normal corticosterone level” refer to the average level of cortisol or corticosterone as determined by measurements of samples (e.g., serum samples) obtained from multiple normal subjects.

Blood levels of cortisol vary in humans during the day and night. Normal morning cortisol values (e.g., for a blood sample taken at about 8 AM) are about 5 micrograms per deciliter (mcg/dL) to about 25 mcg/dL (or about 138-140 nmol/L to about 690-700 nmol/L). Normal late afternoon cortisol values (e.g. for blood samples obtained around 4 PM) may range from about 3 mcg/dL to about 10 mcg/dL (or about 83-84 nmol/L to about 275-280 nmol/L). Normal values depend on the time of day, and may depend on the laboratory making the measurement and the clinical context.

As used herein, a “blood sample” may be a whole blood sample, serum sample, plasma sample, or blood cell sample as appropriate for measuring an analyte level by art-known methods according to conventional use. Similarly, “blood level” of a particular analyte may be the level of the analyte in the whole blood, serum, plasma, or blood cells. For example, the blood level of cortisol or corticosterone may be the level of that analyte in a serum or plasma sample taken from a subject being tested.

The term “average,” refers to value obtained by summing the values obtained from a number of measurements, divided by that number of measurements. The number of measurements may be any number greater than one; however, preferred numbers of measurements may be, e.g., 3, 4, 5, 7, 10, 20, 25, 50, or more.

The term “about” when used in reference to a pre-determined value denotes a range encompassing ±10% of the pre-determined value.

The term “glucocorticoid receptor modulator” (GRM) refers to any compound which modulates GC binding to GR, or which modulates any biological response associated with the binding of GR to an agonist. For example, a GRM that acts as an agonist, such as dexamethasone, increases the activity of tyrosine aminotransferase (TAT) in HepG2 cells (a human liver hepatocellular carcinoma cell line; ECACC, UK). A GRM that acts as an antagonist, such as mifepristone, decreases the activity of tyrosine aminotransferase (TAT) in HepG2 cells. TAT activity can be measured as outlined in the literature by A. Ali et al., J. Med. Chem., 2004, 47, 2441-2452.

As used herein, the term “selective glucocorticoid receptor modulator” (SGRM) refers to any composition or compound which modulates GC binding to GR, or modulates any biological response associated with the binding of a GR to an agonist. By “selective,” the drug preferentially binds to the GR rather than other nuclear receptors, such as the progesterone receptor (PR), the mineralocorticoid receptor (MR) or the androgen receptor (AR). It is preferred that the selective glucocorticoid receptor modulator bind GR with an affinity that is 10× greater ( 1/10^{th} the K_{d} value) than its affinity to the MR, AR, or PR, both the MR and PR, both the MR and AR, both the AR and PR, or to the MR, AR, and PR. In a more preferred embodiment, the selective glucocorticoid receptor modulator binds GR with an affinity that is 100× greater ( 1/100^{th} the K_{d} value) than its affinity to the MR, AR, or PR, both the MR and PR, both the MR and AR, both the AR and PR, or to the MR, AR, and PR. In another embodiment, the selective glucocorticoid receptor modulator binds GR with an affinity that is 1000× greater ( 1/1000^{th} the K_{d} value) than its affinity to the MR, AR, or PR, both the MR and PR, both the MR and AR, both the AR and PR, or to the MR, AR, and PR. CORT113176 and zavacorilant are SGRMs.

“Glucocorticoid receptor antagonist” (GRA) refers to any compound which inhibits GC binding to GR, or which inhibits any biological response associated with the binding of GR to an agonist. Accordingly, GRMs can be identified by measuring the ability of a compound to inhibit the effect of dexamethasone. TAT activity can be measured as outlined in the literature by A. Ali et al., J. Med. Chem., 2004, 47, 2441-2452. A GRA is a compound with an IC₅₀ (half maximal inhibition concentration) of less than 10 micromolar. See Example 1 of U.S. Pat. No. 8,859,774, the entire contents of which is hereby incorporated by reference in its entirety.

As used herein, the term “selective glucocorticoid receptor antagonist” (SGRA) refers to any composition or compound which inhibits GC binding to GR, or which inhibits any biological response associated with the binding of a GR to an agonist (where inhibition is determined with respect to the response in the absence of the compound). By “selective,” the drug preferentially binds to the GR rather than other nuclear receptors, such as the progesterone receptor (PR), the mineralocorticoid receptor (MR) or the androgen receptor (AR). It is preferred that the selective glucocorticoid receptor antagonist bind GR with an affinity that is 10× greater ( 1/10^{th} the K_{d} value) than its affinity to the MR, AR, or PR, both the MR and PR, both the MR and AR, both the AR and PR, or to the MR, AR, and PR. In a more preferred embodiment, the selective glucocorticoid receptor antagonist binds GR with an affinity that is 100× greater ( 1/100^{th} the K_{d} value) than its affinity to the MR, AR, or PR, both the MR and PR, both the MR and AR, both the AR and PR, or to the MR, AR, and PR. In another embodiment, the selective glucocorticoid receptor antagonist binds GR with an affinity that is 1000× greater ( 1/1000^{th} the K_{d} value) than its affinity to the MR, AR, or PR, both the MR and PR, both the MR and AR, both the AR and PR, or to the MR, AR, and PR. CORT113176 and zavacorilant are SGRAs.

As used herein, nonsteroidal GRA, SGRA, GRM, and SGRM compounds include compounds comprising a heteroaryl-ketone fused azadecalin structure (which may also be termed a heteroaryl-ketone fused azadecalin backbone) and compounds comprising an octahydro fused azadecalin structure (which may also be termed an octahydro fused azadecalin backbone).

Exemplary nonsteroidal GRA, SGRA, GRM, and SGRM compounds comprising a heteroaryl-ketone fused azadecalin structure include those described in U.S. Pat. No. 8,859,774. Exemplary heteroaryl-ketone fused azadecalin GR modulator compounds, some of which may act as GRMs, are described in U.S. Pat. No. 8,859,774; in U.S. Pat. No. 9,273,047; in U.S. Pat. No. 9,707,223; and in U.S. Pat. No. 9,956,216, all of which patents are hereby incorporated by reference in their entireties. Exemplary nonsteroidal GRA, SGRA, GRM, and SGRM compounds comprising an octahdro fused azadecalin structure include those described in U.S. Pat. Nos. 10,047,082; 10,323,034; 10,787,449; 11,370,789; and 11,560,379, all of which patents are hereby incorporated by reference in their entireties.

Exemplary GRMs comprising a heteroaryl ketone fused azadecalin structure include those described in U.S. Pat. No. 8,859,774, which can be prepared as disclosed therein, and which patent is incorporated herein in its entirety. Such exemplary GRMs may be SGRMs. In some cases, the GRM comprising a heteroaryl ketone fused azadecalin structure has the following structure:

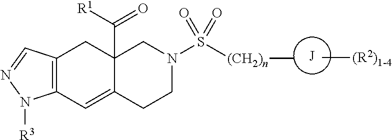

wherein
R¹ is a heteroaryl ring having from 5 to 6 ring members and from 1 to 4 heteroatoms each independently selected from the group consisting of N, O and S, optionally substituted with 1-4 groups each independently selected from R^{1a};
each R^{1a} is independently selected from the group consisting of hydrogen, C₁₋₆ alkyl, halogen, C₁₋₆ haloalkyl, C₁₋₆ alkoxy, C₁₋₆ haloalkoxy, —CN, N-oxide, C₃₋₈ cycloalkyl, and C₃₋₈ heterocycloalkyl;
ring J is selected from the group consisting of a cycloalkyl ring, a heterocycloalkyl ring, an aryl ring and a heteroaryl ring, wherein the heterocycloalkyl and heteroaryl rings have from 5 to 6 ring members and from 1 to 4 heteroatoms each independently selected from the group consisting of N, O and S;
each R² is independently selected from the group consisting of hydrogen, C₁₋₆ alkyl, halogen, C₁₋₆ haloalkyl, C₁₋₆ alkoxy, C₁₋₆ haloalkoxy, C₁₋₆ alkyl-C₁₋₆ alkoxy, —CN, —OH, —NR^{2a}R^{2b}, —C(O)R^{2a}, —C(O)OR^{2a}, —C(O)NR^{2a}R^{2b}, —SR^{2a}, —S(O)R^{2a}, —S(O)₂R^{2a}, C₃₋₈ cycloalkyl, and C₃₋₈ heterocycloalkyl, wherein the heterocycloalkyl groups are optionally substituted with 1-4 R^{2c} groups;
alternatively, two R² groups linked to the same carbon are combined to form an oxo group (═O);
alternatively, two R² groups are combined to form a heterocycloalkyl ring having from 5 to 6 ring members and from 1 to 3 heteroatoms each independently selected from the group consisting of N, O and S, wherein the heterocycloalkyl ring is optionally substituted with from 1 to 3 R^{2d} groups;
R^{2a} and R^{2b} are each independently selected from the group consisting of hydrogen and C₁₋₆ alkyl;
each R^{2c} is independently selected from the group consisting of hydrogen, halogen, hydroxy, C₁₋₆ alkoxy, C₁₋₆ haloalkoxy, —CN, and —NR^{2a}R^{2b};
each R^{2d} is independently selected from the group consisting of hydrogen and C₁₋₆ alkyl, or two R^{2d} groups attached to the same ring atom are combined to form (═O);
R³ is selected from the group consisting of phenyl and pyridyl, each optionally substituted with 1-4 R^{3a} groups;
each R^{3a} is independently selected from the group consisting of hydrogen, halogen, and C₁₋₆ haloalkyl; and
subscript n is an integer from 0 to 3;
or salts and isomers thereof.

In embodiments, that GRM is the compound (R)-(1-(4-fluorophenyl)-6-((4-(trifluoromethyl)phenyl) sulfonyl)-4, 4a, 5,6,7,8-hexahydro-1-H-pyrazolo P,4-g]isoquinolin-4a-yl) (pyridin-2-yl)methanone (termed “dazucorilant” or “CORT113176”), which has the following structure:

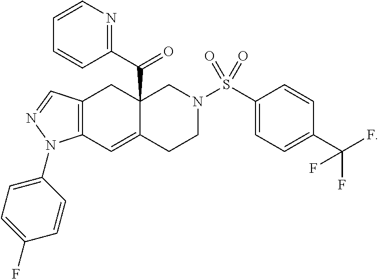 [SMILES: O=C(c1ccccn1)[C@]12Cc3cnn(-c4ccc(F)cc4)c3C=C1CCN(S(=O)(=O)c1ccc(C(F)(F)F)cc1)C2]

Other GRMs comprising a heteroaryl ketone fused azadecalin structure suitable for use in the methods, uses, and compositions disclosed herein include, for example, the compound (R)-(1-(4-fluorophenyl)-6-((1-methyl-1H-pyrazol-4-yl)sulfonyl)-4,4a,5,6,7,8-hexahydro-1H-pyrazolo[3,4-g]isoquinolin-4a-yl)(4-(trifluoromethyl)pyridine-2-yl)methanone (termed “relacorilant” or “CORT125134”), which has the following structure:

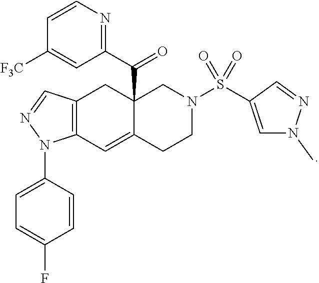 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

Exemplary GRMs comprising an octahydro fused azadecalin structure include those described in U.S. Pat. No. 10,047,082, which can be prepared as disclosed therein, and which patent is incorporated herein in its entirety. Such exemplary GRMs may be SGRMs. In some cases, the GRM comprising an octahydro fused azadecalin structure has the following structure:

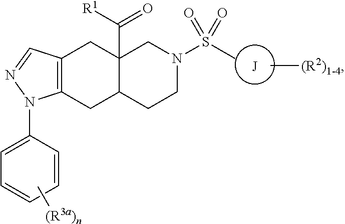

wherein R¹ is a heteroaryl ring having from 5 to 6 ring members and from 1 to 4 heteroatoms each independently selected from the group consisting of N, O and S, optionally substituted with 1-4 groups each independently selected from R^{1a}; each R^{1a} is independently selected from the group consisting of hydrogen, C₁₋₆ alkyl, halogen, C₁₋₆ haloalkyl, C₁₋₆ alkoxy, C₁₋₆ haloalkoxy, N-oxide, and C₃₋₈ cycloalkyl; ring J is selected from the group consisting of an aryl ring and a heteroaryl ring having from 5 to 6 ring members and from 1 to 4 heteroatoms each independently selected from the group consisting of N, O and S; each R² is independently selected from the group consisting of hydrogen, C₁₋₆ alkyl, halogen, C₁₋₆ haloalkyl, C₁₋₆ alkoxy, C₁₋₆ haloalkoxy, C₁₋₆ alkyl-C₁₋₆ alkoxy, CN, OH, NR^{2a}R^{2b}, C(O)R^{2a}, C(O)OR^{2a}, C(O)NR^{2a}R^{2b}, SR^{2a}, S(O)R^{2a}, S(O)₂R^{2a}, C₃₋₈ cycloalkyl, and C₃₋₈ heterocycloalkyl having from 1 to 3 heteroatoms each independently selected from the group consisting of N, O and S; alternatively, two R² groups on adjacent ring atoms are combined to form a heterocycloalkyl ring having from 5 to 6 ring members and from 1 to 3 heteroatoms each independently selected from the group consisting of N, O and S, wherein the heterocycloalkyl ring is optionally substituted with from 1 to 3 R^{2c} groups; R^{2a}, R^{2b} and R^{2c} are each independently selected from the group consisting of hydrogen and C₁₋₆ alkyl; each R^{3a} is independently halogen; and subscript n is an integer from 0 to 3, or salts and isomers thereof.

In embodiments, the octahydro fused azadecalin non-steroidal glucocorticoid receptor modulator is the compound ((4aR,8aS)-1-(4-fluorophenyl)-6-((2-isopropyl-2H-1,2,3-triazol-4-yl)sulfonyl)-4,4a,5,6,7,8,8a,9-octahydro-1H-pyrazolo[3,4-g]isoquinolin-4a-yl)(thiazol-4-yl)methanone (termed zavacorilant, or “CORT125329”), which has the following structure:

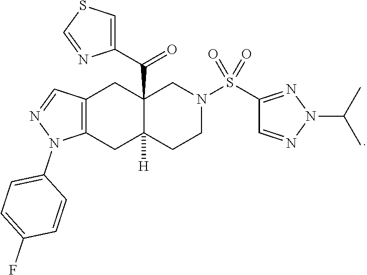 [SMILES: CC(C)n1ncc(S(=O)(=O)N2CC[C@H]3Cc4c(cnn4-c4ccc(F)cc4)C[C@]3(C(=O)c3cscn3)C2)n1]

Other GRMs comprising an octahydro fused azadecalin structure suitable for use in the methods, uses, and compositions disclosed herein include, for example, the compound ((4aR,8aS)-1-(4-fluorophenyl)-6-((2-methyl-2H-1,2,3-triazol-4-yl)sulfonyl)-4,4a,5,6,7,8,8a,9-octahydro-1H-pyrazolo[3,4-g]isoquinolin-4a-yl)(4-(trifluoromethyl)pyridin-2-yl)methanone (termed “exicorilant” or “CORT125281”), which has the following structure:

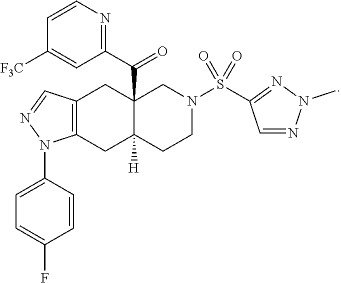 [SMILES: Cn1ncc(S(=O)(=O)N2CC[C@H]3Cc4c(cnn4-c4ccc(F)cc4)C[C@]3(C(=O)c3cc(C(F)(F)F)ccn3)C2)n1]

As used herein, the term “composition” is intended to encompass a product comprising the specified ingredients such as the said compounds, their tautomeric forms, their derivatives, their analogues, their stereoisomers, their polymorphs, their deuterated species, their pharmaceutically acceptable salts, esters, ethers, metabolites, mixtures of isomers, their pharmaceutically acceptable solvates and pharmaceutically acceptable compositions in specified amounts, as well as any product which results, directly or indirectly, from combination of the specified ingredients in the specified amounts. Such term in relation to a pharmaceutical composition is intended to encompass a product comprising the active ingredient (s), and the inert ingredient (s) that make up the carrier, as well as any product which results, directly or indirectly, in combination, complexation or aggregation of any two or more of the ingredients, or from dissociation of one or more of the ingredients, or from other types of reactions or interactions of one or more of the ingredients. Accordingly, the pharmaceutical compositions of the present invention are meant to encompass any composition made by admixing compounds of the present invention and their pharmaceutically acceptable carriers.

In some embodiments, the term “consisting essentially of” refers to a composition in a formulation whose only active ingredient is the indicated active ingredient, however, other compounds may be included which are for stabilizing, preserving, etc. the formulation, but are not involved directly in the therapeutic effect of the indicated active ingredient. In some embodiments, the term “consisting essentially of” can refer to compositions which contain the active ingredient and components which facilitate the release of the active ingredient. For example, the composition can contain one or more components that provide extended release of the active ingredient over time to the subject. In some embodiments, the term “consisting” refers to a composition, which contains the active ingredient and a pharmaceutically acceptable carrier or excipient.

“Salt” refers to acid or base salts of the compounds used in the methods of the present invention. Illustrative examples of pharmaceutically-acceptable salts are mineral acid (hydrochloric acid, hydrobromic acid, phosphoric acid, and the like) salts, organic acid (acetic acid, propionic acid, glutamic acid, citric acid, and the like) salts, and quaternary ammonium (methyl iodide, ethyl iodide, and the like) salts. It is understood that the pharmaceutically-acceptable salts are non-toxic. Additional information on suitable pharmaceutically-acceptable salts can be found in Remington's Pharmaceutical Sciences, 17th ed., Mack Publishing Company, Easton, Pa., 1985, which is incorporated herein by reference.

“Pharmaceutically-acceptable excipient” and “pharmaceutically-acceptable carrier” refer to a substance that aids the administration of an active agent to—and absorption by—a subject and can be included in the compositions of the present invention without causing a significant adverse toxicological effect on the patient. As used herein, these terms are intended to include any and all solvents, dispersion media, coatings, antibacterial and antifungal agents, antioxidant agents, isotonic and absorption delaying agents, and the like, compatible with pharmaceutical administration. Non-limiting examples of pharmaceutically-acceptable excipients include water, NaCl, normal saline solutions, lactated Ringer's, normal sucrose, normal glucose, binders, fillers, disintegrants, encapsulating agents, plasticizers, lubricants, coatings, sweeteners, flavors and colors, and the like. One of ordinary skill in the art will recognize that other pharmaceutical excipients are useful in the present invention. The use of such media and agents for pharmaceutically active substances is well known in the art. Except insofar as any conventional media or agent is incompatible with the active compound, use thereof in the compositions is contemplated. Supplementary active compounds can also be incorporated into the compositions. One of ordinary skill in the art will recognize that other pharmaceutical excipients are useful in the present invention.

### Pharmaceutical Compositions and Administration

In embodiments, the present invention provides a pharmaceutical composition for treating Huntington's Disease (HD), the pharmaceutical composition including a pharmaceutically acceptable excipient and a GRM. In some embodiments, the pharmaceutical composition includes a pharmaceutically acceptable excipient and a SGRM. In preferred embodiments, the pharmaceutical composition includes a pharmaceutically acceptable excipient and a nonsterodial SGRM having a heteroaryl-ketone fused azadecalin structure or an octahydro fused azadecalin structure.

GRMs and SGRMs (as used herein, GRMs and SGRMs include nonsteroidal GRMs and nonsteroidal SGRMS), can be prepared and administered in a wide variety of oral, parenteral and topical dosage forms. Oral preparations include tablets, pills, powder, dragees, capsules, liquids, lozenges, gels, syrups, slurries, suspensions, etc., suitable for ingestion by the patient. GRMs and SGRMs can also be administered by injection, that is, intravenously, intramuscularly, intracutaneously, subcutaneously, intraduodenally, or intraperitoneally. Also, GRMs and SGRMs can be administered by inhalation, for example, intranasally. Additionally, GRMs and SGRMs can be administered transdermally. Accordingly, the present invention also provides pharmaceutical compositions including a pharmaceutically acceptable carrier or excipient and a GRM or SGRM.

For preparing pharmaceutical compositions from GRMs and SGRMs, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Mack Publishing Co, Easton PA (“Remington's”).

In powders, the carrier is a finely divided solid, which is in a mixture with the finely divided active component, a GRM or SGRM. In tablets, the active component is mixed with the carrier having the necessary binding properties in suitable proportions and compacted in the shape and size desired.

The powders and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients are carbohydrate or protein fillers include, but are not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain GR modulator mixed with a filler or binders such as lactose or starches, lubricants such as talc or magnesium stearate, and, optionally, stabilizers. In soft capsules, the GR modulator compounds may be dissolved or suspended in suitable liquids, such as fatty oils, liquid paraffin, or liquid polyethylene glycol with or without stabilizers.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending a SGRM in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

GRMs and SGRMs can be delivered by transdermally, by a topical route, formulated as applicator sticks, solutions, suspensions, emulsions, gels, creams, ointments, pastes, jellies, paints, powders, and aerosols.

GRMs and SGRMs can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

In some cases, the pharmaceutical formulations of the invention can be provided as a salt and can be formed with many acids, including but not limited to hydrochloric, sulfuric, acetic, lactic, tartaric, malic, succinic, etc. Salts tend to be more soluble in aqueous or other protonic solvents than are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use

In another embodiment, the formulations of the invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the GR modulator into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The pharmaceutical preparation is preferably in unit dosage form. In such form the preparation is subdivided into unit doses containing appropriate quantities of the active component, a GRM or SGRM. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, or from 1.0 mg to 6000 mg, or from 5 mg to 5000 mg, or from 10 mg to 2000 mg, or from 15 mg to 1500 mg, or from 20 mg to 1250 mg, or from 25 mg to 1000 mg, or from 50 mg to 750 mg. Suitable dosages also include about 1 mg, 5, 10, 20, 25, 30, 40, 50, 60, 70, 75, 80, 90, 100, 150, 200, 225, 300, 375, 400, 450, 500, 550, 600, 700, 800, 900, 1000, 1100, 1200, 1300, 1400, 1500, 1600, 1700, 1800, 1900, or 2000 mg, according to the particular application and the potency of the active component. The composition can, if desired, also contain other compatible therapeutic agents.

The pharmaceutical preparation is preferably in unit dosage form. In such form the preparation is subdivided into unit doses containing appropriate quantities of the compounds and compositions of the present invention. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

GRMs can be administered orally. For example, the GRM can be administered as a pill, a capsule, or liquid formulation as described herein. Alternatively, GRMs can be provided via parenteral administration. For example, the GRM can be administered intravenously (e.g., by injection or infusion). Additional methods of administration of the compounds described herein, and pharmaceutical compositions or formulations thereof, are described herein.

In some embodiments, the GRM is administered in one dose. In other embodiments, the GRM is administered in more than one dose, e.g., 2 doses, 3 doses, 4 doses, 5 doses, 6 doses, 7 doses, or more. In some cases, the doses are of an equivalent amount. In other cases, the doses are of different amounts. The doses can increase or taper over the duration of administration. The amount will vary according to, for example, the GRM properties and patient characteristics.

Any suitable GRM dose may be used in the methods disclosed herein. The dose of GRM that is administered can be at least about 10 milligrams (mg) per day (mg/day), or about 15 mg/day, or about 20 mg/day, or about 25 mg/day, or about 35 mg/day, or about 45 mg/day, or about 50 mg/day, or about 75 mg/day, or about 100 mg/day, or about 125 mg/day, or about 150 mg/day, or about 175 mg/day, or about 200 mg/day, or about 225 mg/day, or about 250 mg/day, or about 300 mg/day, or about 350 mg/day, or about 375 mg/day, or about 400 mg/day, or about 450 mg/day, or about 500 mg/day, or about 525 mg/day, or about 550 mg/day, or about 600 mg/day, or about 675 mg/day, or about 700 mg/day, or about 750 mg/day, or about 800 mg/day, or about 825 mg/day, or about 900 mg/day, or about 975 mg/day, or about 1000 mg/day, or about 1050 mg/day, or about 1100 mg/day, or about 1200 mg/day, or more. In embodiments, the GRM is administered orally. In some embodiments, the GRM is administered in at least one dose. In other words, the GRM can be administered in 1, 2, 3, 4, 5, 6, 7, 8, 9, 10 or more doses. In embodiments, the GRM is administered orally in 1, 2, 3, 4, 5, 6, 7, 8, 9, 10 or more doses.

The subject may be administered at least one dose of GRM in one or more doses over, for example, a 2-48 hour period. In some embodiments, the GRM is administered as a single dose. In other embodiments, the GRM is administered in more than one dose, e.g. 2 doses, 3 doses, 4 doses, 5 doses, or more doses over a 2-48 hour period, e.g., a 2 hour period, a 3 hour period, a 4 hour period, a 5 hour period, a 6 hour period, a 7 hour period, a 8 hour period, a 9 hour period, a 10 hour period, a 11 hour period, a 12 hour period, a 14 hour period, a 16 hour period, a 18 hour period, a 20 hour period, a 22 hour period, a 24 hour period, a 26 hour period, a 28 hour period, a 30 hour period, a 32 hour period, a 34 hour period, a 36 hour period, a 38 hour period, a 40 hour period, a 42 hour period, a 44 hour period, a 46 hour period or a 48 hour period. In some embodiments, the GRM is administered over 2-48 hours, 2-36 hours, 2-24 hours, 2-12 hours, 2-8 hours, 8-12 hours, 8-24 hours, 8-36 hours, 8-48 hours, 9-36 hours, 9-24 hours, 9-20 hours, 9-12 hours, 12-48 hours, 12-36 hours, 12-24 hours, 18-48 hours, 18-36 hours, 18-24 hours, 24-36 hours, 24-48 hours, 36-48 hours, or 42-48 hours.

Single or multiple administrations of formulations can be administered depending on the dosage and frequency as required and tolerated by the patient. The formulations should provide a sufficient quantity of active agent to effectively treat the disease state. Thus, in one embodiment, the pharmaceutical formulation for oral administration of a GRM is in a daily amount of between about 0.01 to about 150 mg per kilogram of body weight per day (mg/kg/day). In some embodiments, the daily amount is from about 0.1 to about 50 mg/kg/day, or about 0.5 to about 35 mg/kg/day, or about 1 to about 25 mg/kg/day, or about 2 to about 20 mg/kg/day. In embodiments, the daily dose of the GRM is between about 10 milligrams per day (mg/day) and about 1200 mg/day, or between about 15 mg/day to about 1100 mg/day, or between about 20 mg/day to about 1000 mg/day, or between about 25 mg/day to about 975 mg/day, or between about 50 mg/day to about 900 mg/day, or between about 75 mg/day and about 825 mg/day, or between about 100 mg/day to about 800 mg/day, or between about 125 mg/day to about 750 mg/day, or between about 150 mg/day to about 500 mg/day.

In embodiments, the GRM is administered orally to the patient. In embodiments, the GRM is administrated for at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or 80 weeks. In embodiments, the GRM may be administered to the patient for longer than 80 weeks, or even longer.

In some embodiments, administration of a GRM or SGRM is not continuous and can be stopped for one or more periods of time, followed by one or more periods of time where administration resumes. Suitable periods where administration stops include 5 to 9 weeks, 5 to 16 weeks, 9 to 16 weeks, 16 to 24 weeks, 16 to 32 weeks, 24 to 32 weeks, 24 to 48 weeks, 32 to 48 weeks, 32 to 52 weeks, 48 to 52 weeks, 48 to 64 weeks, 52 to 64 weeks, 52 to 72 weeks, 64 to 72 weeks, 64 to 80 weeks, 72 to 80 weeks, 72 to 88 weeks, 80 to 88 weeks, 80 to 96 weeks, 88 to 96 weeks, and 96 to 100 weeks. Suitable periods where administration stops also include 5, 6, 7, 8, 9, 10, 11, 12, 13, 14, 15, 16, 17, 18, 19, 20, 24, 25, 30, 32, 35, 40, 45, 48 50, 52, 55, 60, 64, 65, 68, 70, 72, 75, 80, 85, 88 90, 95, 96, and 100 weeks.

The dosage regimen also takes into consideration pharmacokinetics parameters well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, GR modulator and disease or condition treated.

SGRMs can be used in combination with other active agents known to be useful in modulating a glucocorticoid receptor, or with adjunctive agents that may not be effective alone, but may contribute to the efficacy of the active agent.

After a pharmaceutical composition including a GRM or SGRM has been formulated in an acceptable carrier, it can be placed in an appropriate container and labeled for treatment of an indicated condition. For administration of a GRM or SGRM, such labeling would include, e.g., instructions concerning the amount, frequency and method of administration.

In another embodiment, the compositions of the present invention are useful for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

### I. Combination Therapies

Administration of the therapeutic compounds or agents to a patient will follow general protocols for the administration of such compounds, taking into account the toxicity, if any, of the therapy. The present methods can be combined with other means of treatment.

Various combinations with a GRM or SGRM and another agent for treating Huntington's Disease (or a combination of such agents and compounds) may be employed to treat the patient. For example, tetrabenazine may be prescribed for a patient suffering from HD; other drugs sometimes administered to patients suffering from HD include, e.g., haloperidol, risperidone, chlorpromazine, and amantadine. By “combination therapy” or “in combination with”, it is not intended to imply that the therapeutic agents must be administered at the same time and/or formulated for delivery together, although these methods of delivery are within the scope described herein. The GRM or SGRM and the other agent can be administered following the same or different dosing regimen. In some embodiments, the GRM or SGRM and the other agent is administered sequentially in any order during the entire or portions of the treatment period. In some embodiments, the GRM or SGRM and the other agent is administered simultaneously or approximately simultaneously (e.g., within about 1, 5, 10, 15, 20, or 30 minutes of each other). Non-limiting examples of combination therapies are as follows, with administration of the GRM or SGRM and another therapeutic agent for example, GRM or SGRM is “A” and the other therapeutic agent is “B”:

A/B/AB/A/BB/B/AA/A/BA/B/BB/A/AA/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A.

### EXAMPLES

The following examples are provided by way of illustration only and not by way of limitation. Those of skill will readily recognize a variety of noncritical parameters which could be changed or modified to yield essentially similar results.

### Use of Glucocorticoid Receptor Modulator Cort113176 to Treat Motor and Neuropathological Symptoms of Huntington's Disease in R6/2 Mice

Huntington's Disease (HD) is a genetic neurodegenerative disease caused by a mutation in the huntingtin gene. Mutant huntingtin (mHtt) leads to cellular malfunction, protein aggregates and eventually results in neuronal cell death. Patients with HD show impaired motor functions and cognitive decline. Elevated levels of glucocorticoids have been found in patients with HD and in HD mouse models.

The glucocorticoid receptor modulators (GRM) CORT113176 (also known as dazucorilant) and zavacorilant lack cross-reactivity with other steroid receptors. CORT113176 has been proven to be effective in models for Alzheimer's disease and amyotrophic lateral sclerosis (ALS)⁷⁻¹⁰.

We evaluated the efficacy of the selective GRM CORT113176 to attenuate the symptoms of HD in the commonly used R6/2 mouse model, characterized by severe motor decline in the course of weeks. The results of these studies showed that treatment with CORT113176 delayed several motor symptoms in male mice, but less so in female HD mice. CORT113176 reduced the number of epileptic seizures observed in mice of both sexes (no seizures were observed in female mice treated with CORT113176). Region-specific changes on glial cells in HD mice were normalized by CORT113176 treatment. Finally, treatment with CORT113176 reduced the formation of hippocampal CA1 and striatal mHtt aggregates.

Material and Methods Used in this Study

### Animals

HD and wild-type (WT) R6/2 mice were purchased from JAX (The Jackson Laboratory, ME, USA) and arrived in the animal facility at 4-weeks of age. The mice were housed under normal conditions (room temperature, 12 hours light/dark cycle) with food and water ab libitum. The mice were housed 3-4 per cage, separated by sex, but different genotypes were housed together to improve the survival of the HD mice¹¹. Genotyping on tail samples that were collected post-mortem was performed by Laragen (CA, USA) to confirm that all HD mice had between 120-130 CAG repeats.

Animal studies: experimental procedures From 6 weeks of age onwards, mice received a daily subcutaneous (s.c.) injection either with 30 mg/kg of CORT113176 or vehicle (Veh; 10% ethanol and castor oil in experiment 1 or sesame oil in experiment 2) for five consecutive weeks. In experiment 1, male and female mice were assigned into one of the following groups: 1) WT+Veh (n=5 per sex), 2) WT+CORT113176 (n=5), 3) HD+Veh (n=5) or 4) HD+CORT113176 (n=5). In the final analysis there was an unequal group distribution for the male mice due to a mislabeled genotype and a mouse reaching an earlier endpoint resulting in n=6 in the WT+Veh group and n=3 in the HD+Veh group. In experiment 2, male mice were assigned into the following groups: 1) WT+Veh (n=10), 2) HD+Veh (n=10, ultimately n=8 due to a non-responder that met outlier criteria and a mouse reaching an earlier timepoint) and 3) HD+CORT113176 (n=10). Bodyweight was measured weekly and motor tests were performed at 1 PM to monitor the disease progression (as described below). On day 19 and day 34, blood samples were taken in the morning to evaluate corticosterone levels. On day 34, the animals were sacrificed using CO₂ inhalation, blood was collected via cardiac puncture and the animals were perfused with cold PBS. Afterwards, several tissues were harvested and snap-frozen for further analysis. Brains were collected and stored for 24 hours at 4% PFA for IHC (further described below).

### Motor Function Studies

Grip strength: Grip strength measurements were performed weekly to quantify muscle strength. For this procedure. mice were lifted by their tail in front of a metal bar that was attached to a Chatillon Grip Force Meter (Columbus Instruments, OH, USA) and mice were allowed to grasp the grid tightly with both forepaws. The mouse was then gently pulled away until grip was lost, and the force that was required to do this was monitored. This test was repeated 15 times with short resting periods between every 5 measurements¹². The three highest scores were used for the analysis.

Clasping test: the clasping test was performed weekly to assess the degradation of the cortico-striatal pathway, the primary input circuitry of the basal ganglia that controls many processes including voluntary movement and motor coordination¹³. For this procedure, the mice were lifted by their tail to a height of approximately 50 cm for no longer than 10 seconds. During this procedure, the hindlimbs were observed and a score was given based on their position in relation to the abdomen ranging from 0-3¹⁴. This was repeated three times and the average of the scores was used for the analysis.

Epileptic seizures: several instances of (spontaneous) seizures were observed in the HD mice during the study. These seizures were not anticipated, but every case was documented throughout the experiment and scored using the Racine scale¹⁵.

Immunohistochemistry Brains were isolated and the left hemisphere was fixated in 4% PFA (Sigma-Aldrich, 8187085000) for 24 hours. Afterwards the brains were transferred to a 30% sucrose solution for 24-48 hours and stored at −80° C. until further processing. Cryosections of 10 μM were collected (CryoStar NX70) on slides (Avantor, VWR® Microscope Slides, 631-1166) and stored at −20° C. until staining. The slides were incubated for 25 minutes with 0.1% Triton X-100 (Sigma-Aldrich), washed with 0.1% PBS/Tween (Tween®20, Sigma-Aldrich). The background signal was blocked with 5% PBSA for 30 minutes before the incubation with the primary antibody DARP32, (1:500, abcam, EP720Y, ab40801), GFAP (1:500, Agilent DAKO, Z0334), Iba1, (1:500, Fujifilm Wako, 019-19741) or mHtt, 1:500 (1:500, abcam, EPR5526, ab209668) overnight at 4° C. The following day slides were washed with 0.1% PBS/Tween before incubation with the secondary antibody goat anti-rabbit Alexa Fluor 488 (1:250, Invitrogen, A-110088) for 30 minutes. After a final wash with 0.1% PBS/Tween, ProLong gold with DAPI (Invitrogen, P36931) was added with a coverslip (Thermo-Fisher, Menzel-Glaser, 0980) and the slides were dried at room temperature. The slides of DARP32, GFAP and Iba1 were scanned with the Axio Slide Scanner (Zeiss, Axio Slide Scan.Z1) and the mHtt slides were analyzed using the confocal microscope (Leica, White Light Laser Confocal Microscope TCS SP8 X). All obtained images were analyzed using ImageJ (version 1.52p). DARP32, GFAP and Iba1 the mean intensity was measured in the striatum and hippocampus. The total number of aggregates in a specific region of interest (ROI), the total area in the ROI and the average size of the aggregates were measured.

Gene expression Frozen tissues were homogenized in Lysing Matrix D Minibead tubes (MP Biomedicals, #116913500) using TriPure isolation reagent (Roche, #11667165001) and total RNA was isolated following the protocol provided by the manufacturer. cDNA was synthesized using M-MLV reverse transcriptase (Promega, #M1705). Real-time quantitative PCR was performed using IQ SYBR-Green Supermix (Bio-Rad, #170-8885) and the Bio-Rad CFX96 system.

### Example 1

Data of male and female mice are presented separately, given the substantial differences between the sexes in the response to CORT113176.

### CORT113176 Treatment Partially Rescues Muscle Strength in Male, but not in Female HD Mice

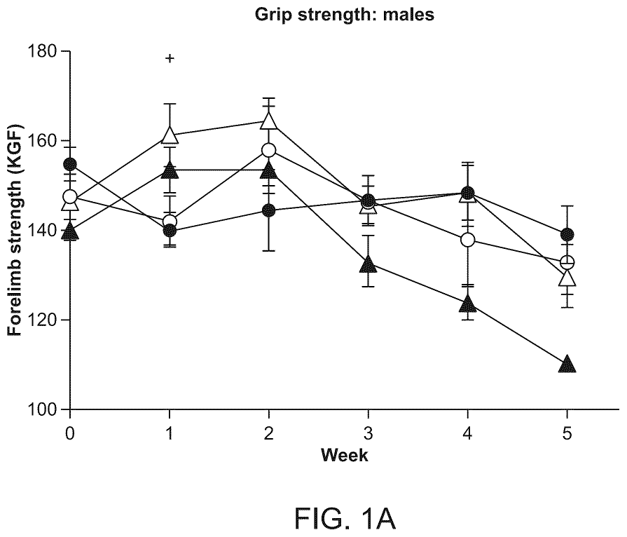

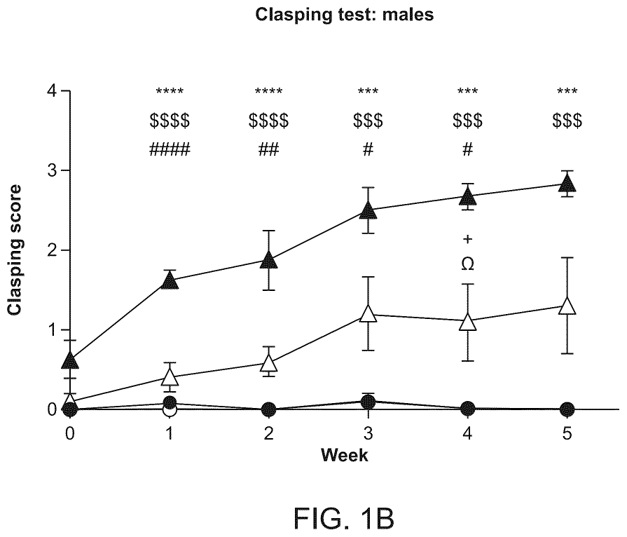

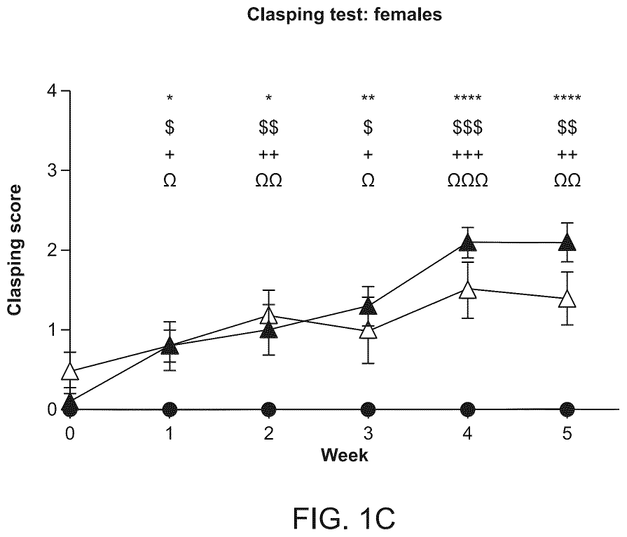

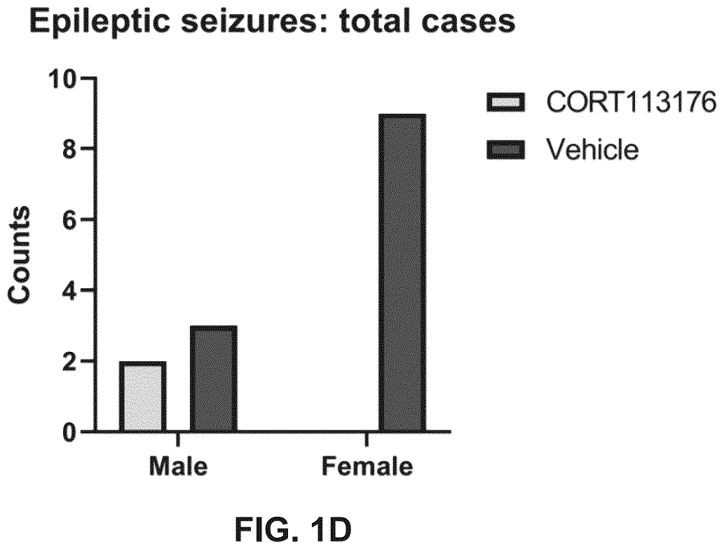

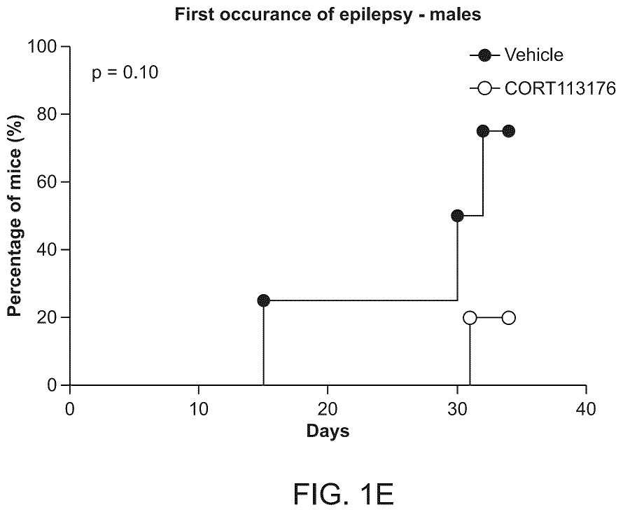

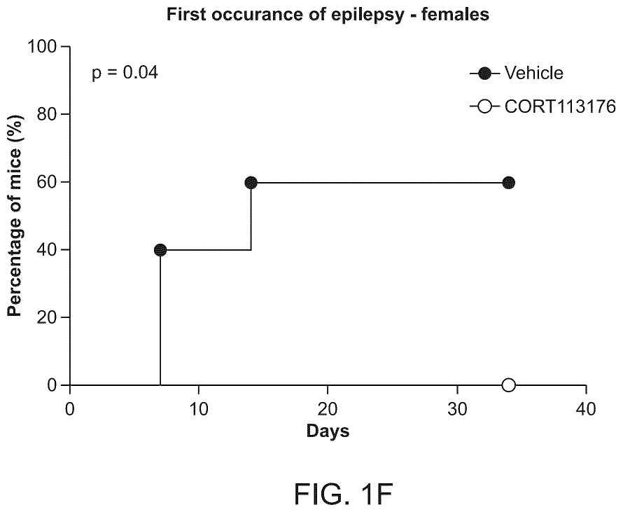

Forelimb grip strength was measured to assess the effect of CORT113176 on muscle function. We observed a genotype effect over time in both male and female mice. Muscle strength in male WT mice remained relatively stable over the course of five weeks (FIG. 1.A). In contrast, vehicle-treated male HD mice showed a steady decline in grip strength over time, starting from week 2. CORT113176 treatment attenuated this decrease of muscle function in male HD mice. In FIGS. 1A, 1B, and 1C filled circles indicate results from wild-type mice treated with vehicle; open circles indicate results from wild-type mice treated with CORT113176; filled triangles indicate results from HD mice treated with vehicle; and open triangles indicate results from HD mice treated with CORT113176.

### CORT113176 Treatment Delays Clasping Score in Male and Female HD Mice

The clasping test was performed as a measure to evaluate the effect of CORT113176 treatment on the functional decline of the cortico-striatal pathway, an important pathway for voluntary movement and motor coordination. A high clasping score indicates loss of voluntary motor coordination and therefore a more progressed HD phenotype. None of the WT mice showed clasping behavior. We observed a genotype effect over time for both sexes.

In male mice, there was a significant interaction effect between HD and CORT113176. Vehicle-treated male HD mice showed an increase in clasping score starting from week 1 as compared to the WT mice (FIG. 1B), and CORT113176 treatment reduced clasping score in HD mice as compared to vehicle-treated HD mice.

In female mice, vehicle-treated HD mice showed increased clasping over time compared to the WT mice (FIG. 1C). There was an interaction for genotype, CORT113176 treatment and time suggesting that at later timepoints the treatment did attenuate the clasping score.

### CORT113176 Treatment Prevents Epileptic Seizures in Both Sexes

Epileptic seizures are a known symptom in Juvenile HD (JHD), a form of HD that affects children and teenagers and this feature is recapitulated in the R6/2 mouse model²⁵. Total observed epileptic seizures during experimental procedures were documented, which were of tonic-clonic nature according to the Racine scale¹⁵. Epileptic seizures occurred most frequently in the vehicle-treated female HD mice, while none were observed in the CORT113176-treated female HD mice (FIG. 1D). Overall, epileptic seizures were less common in the male HD mice, although these also occurred slightly more frequently in the vehicle-treated HD mice as compared to the CORT113176-treated HD male mice (FIG. 1D). The epileptic seizures in the CORT113176-treated HD mice occurred mostly near the end of the five weeks (day 32) while the cases of the vehicle-treated HD animals occurred throughout the whole 5-week study (FIGS. 1E and 1F).

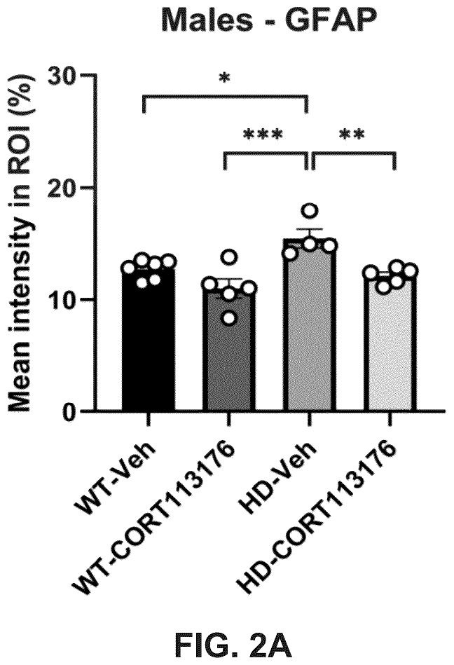

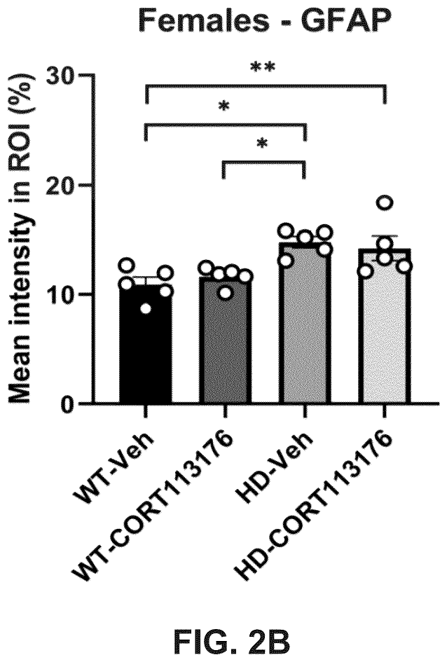

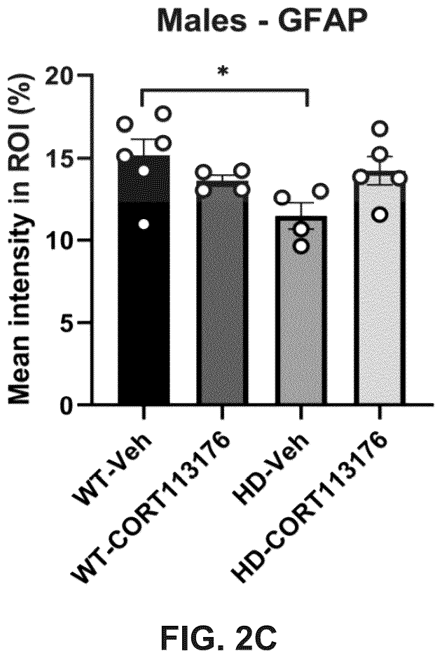

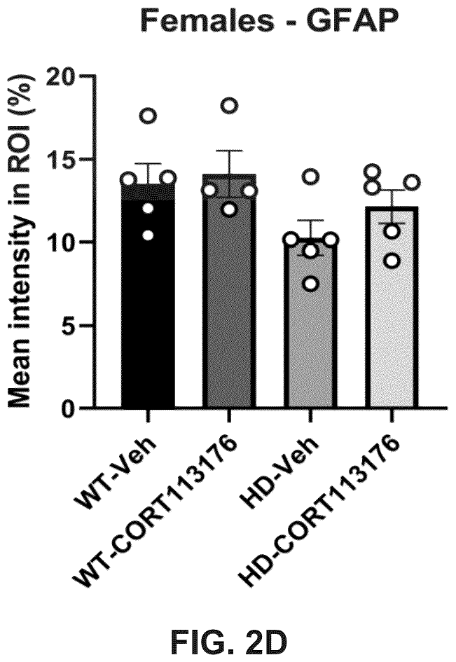

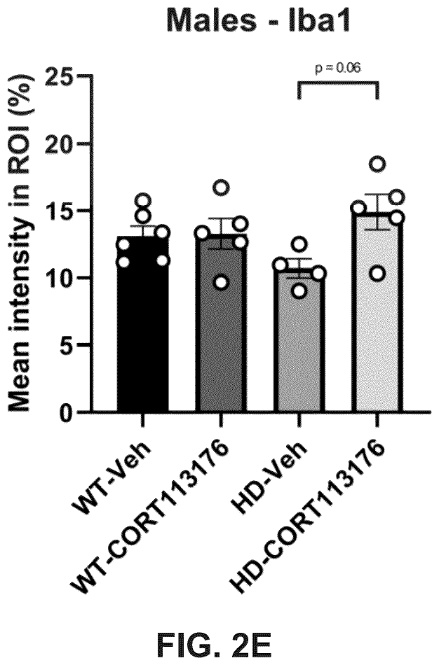

Astrocyte and microglia activity was assessed by GFAP and Iba1 staining, respectively. In the striatum, GFAP immunoreactivity was significantly increased in both male and female HD mice compared to WT mice (FIG. 2A, HD; and 2B HD) and treatment with CORT113176 normalized GFAP levels in male, but not in female mice (FIG. 2A, CORT113176; and 2B). A significant genotype effect was observed for Iba1 immunoreactivity in the striatum of both male and female mice.

Next, we evaluated astrocyte and microglia markers in the hippocampus. Within this brain region, an overall trend for the genotype was observed for GFAP staining (FIG. 2C, HD) and a significant reduction of GFAP staining intensity in the vehicle-treated HD male mice as compared to the WT (FIG. 2C). Similar to the striatum, CORT113176 treatment tended to normalize this HD effect (FIG. 2C). A graphical illustration of GFAP staining intensity in female mice is shown in FIG. 2D. Iba1 intensity in the male HD hippocampus tended to be higher with CORT113176 treatment (FIG. 2E, CORT113176).

### Example 2

The results disclosed in Example 1 demonstrate unanticipated sex differences. In order to replicate and further characterize the potential beneficial effects of CORT113176 treatment in a larger cohort of male R6/2 mice. These results confirmed the positive effects of CORT113176 administration on grip strength, clasping behavior, and epileptic seizures.

### CORT113176 Treatment Delays Several Motor Function Symptoms in Male HD Mice

In line with the results discussed above CORT113176 improved grip strength and reduced clasping score compared with vehicle treated mice. Only one epileptic seizure was observed in the CORT113176-treated HD mice (occurring near the end of the study), while several vehicle-treated HD mice had seizures at earlier times throughout the study.

### CORT113176 Treatment Reduces the Formation of Mutant Huntingtin Aggregates in the Striatum and in the CA1 Region of the Hippocampus

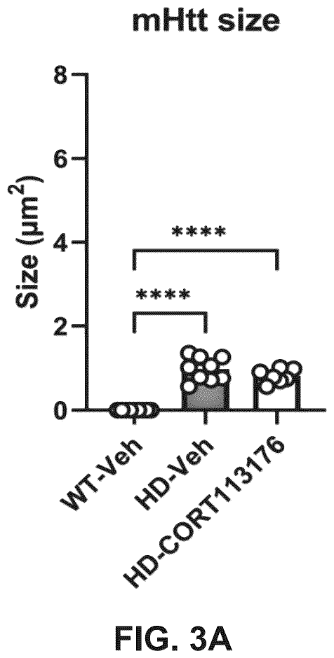

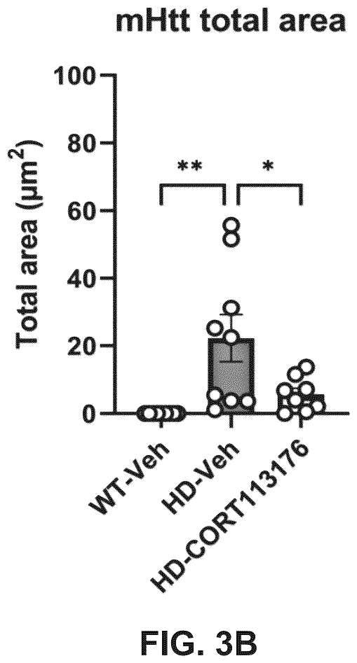

The formation of mHtt aggregates was assessed as these are considered the hallmark of the HD phenotype. In the striatum, vehicle-treated HD mice showed a significant increase in the number of aggregates as compared to the WT mice. Treatment with CORT113176 significantly decreased this number of mHtt aggregates and the aggregates were of a similar size in vehicle- and CORT113176-treated HD groups (FIG. 3A and FIG. 3B).

In the CA1 region of the hippocampus, the total number of aggregates was significantly increased in HD mice compared to WT mice. CORT113176-treated mice had significantly fewer aggregates in this brain region compared to vehicle-treated HD mice. In addition, the CORT113176-treated mice display smaller aggregates compared to the vehicle-treated HD mice resulting in a smaller total aggregate area in the CA1 region.

### DISCUSSION AND SUMMARY

In this study, we evaluated the efficacy of CORT113176, a selective GRM, to attenuate the symptoms of HD in the R6/2 mouse model. Here, we show in two independent studies that CORT11376 delays several motor and neuropathological symptoms of HD in male mice.

In the mice, we observed that the treatment with CORT113176 alleviated several HD-related symptoms. As such, treatment with CORT113176 partially improved grip strength and significantly reduced clasping score. Furthermore, CORT113176 treatment prevented the occurrence of epileptic seizures, commonly found in JHD and the R6/2 model¹⁶-17 CORT113176 also reduced protein aggregation in the brain of male R6/2 mice (female mice not evaluated). Taken together, there was a clear efficacy of CORT113176 in the CNS, particularly in male mice.

All patents, patent publications, publications, and patent applications cited in this specification are hereby incorporated by reference herein in their entireties as if each individual publication or patent application were specifically and individually indicated to be incorporated by reference. In addition, although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

### REFERENCES

1. Vyas S, Rodrigues A J, Silva J M, Tronche F, Almeida O F, Sousa N, et al. Chronic Stress and Glucocorticoids: From Neuronal Plasticity to Neurodegeneration. Neural Plast. 2016; 2016:6391686.
2. Schakman O, Kalista S, Barbe C, Loumaye A, Thissen J P. Glucocorticoid-induced skeletal muscle atrophy. Int J Biochem Cell Biol. 2013; 45(10):2163-72.
3. Vegiopoulos A, Herzig S. Glucocorticoids, metabolism and metabolic diseases. Mol Cell Endocrinol. 2007; 275(1-2):43-61.
4. Bjorkqvist M, Petersen A, Bacos K, Isaacs J, Norlen P, Gil J, et al. Progressive alterations in the hypothalamic-pituitary-adrenal axis in the R6/2 transgenic mouse model of Huntington's disease. Hum Mol Genet. 2006; 15(10):1713-21.
5. Dufour B D, McBride J L. Corticosterone dysregulation exacerbates disease progression in the R6/2 transgenic mouse model of Huntington's disease. Exp Neurol. 2016; 283(Pt A):308-17.
6. Morton A J, Lagan M A, Skepper J N, Dunnett S B. Progressive formation of inclusions in the striatum and hippocampus of mice transgenic for the human Huntington's disease mutation. J Neurocytol. 2000; 29(9):679-702.
7. Hunt H J, Belanoff J K, Golding E, Gourdet B, Phillips T, Swift D, et al. 1H-Pyrazolo[3,4-g]hexahydro-isoquinolines as potent GRMs with reduced hERG inhibition and an improved pharmacokinetic profile. Bioorg Med Chem Lett. 2015; 25(24):5720-5.
8. Pineau F, Canet G, Desrumaux C, Hunt H, Chevallier N, Ollivier M, et al. New selective glucocorticoid receptor modulators reverse amyloid-beta peptide-induced hippocampus toxicity. Neurobiol Aging. 2016; 45:109-22.
9. Meyer M, Lara A, Hunt H, Belanoff J, de Kloet E R, Gonzalez Deniselle M C, et al. The Selective Glucocorticoid Receptor Modulator Cort 113176 Reduces Neurodegeneration and Neuroinflammation in Wobbler Mice Spinal Cord. Neuroscience. 2018; 384:384-96.
10. Meyer M, Kruse M S, Garay L, Lima A, Roig P, Hunt H, et al. Long-term effects of the glucocorticoid receptor modulator CORT113176 in murine motoneuron degeneration. Brain Res. 2020; 1727.
11. Story D, Gallien J, Al-Gharaibeh A, Sandstrom M, Rossignol J, Dunbar G L. Housing R6/2 Mice with Wild-Type Littermates Increases Lifespan. J Huntingtons Dis. 2021; 10(4):455-8.
12. Aartsma-Rus A, van Putten M. Assessing functional performance in the mdx mouse model. J Vis Exp. 2014(85).
13. Haber S N. Corticostriatal circuitry. Dialogues Clin Neurosci. 2016; 18(1):7-21.
14. Guyenet S J, Furrer S A, Damian V M, Baughan T D, La Spada A R, Garden G A. A simple composite phenotype scoring system for evaluating mouse models of cerebellar ataxia. J Vis Exp. 2010(39).
15. Van Erum J, Van Dam D, De Deyn P P. PTZ-induced seizures in mice require a revised Racine scale. Epilepsy Behav. 2019; 95:51-5.
16. Thakor B, Jagtap S A, Joshi A. Juvenile Huntington's disease masquerading as progressive myoclonus epilepsy. Epilepsy Behav Rep. 2021; 16:100470.
17. Cepeda C, Oikonomou K D, Cummings D, Barry J, Yazon V W, Chen D T, et al. Developmental origins of cortical hyperexcitability in Huntington's disease: Review and new observations. J Neurosci Res. 2019; 97(12):1624-35.

1. A method of treating a patient suffering from Huntington's Disease (HD), the method comprising administering an effective amount of a heteroaryl ketone fused azadecalin glucocorticoid receptor modulator (GRM) or an octahydro fused azadecalin GRM to the patient effective to treat HD.

2. The method of claim 1, wherein the GRM is a heteroaryl ketone fused azadecalin GRM.

3. The method of claim 1, wherein the GRM is an octahydro fused azadecalin GRM.

4. The method of claim 1, wherein the treatment is effective to treat a symptom of HD.

5. The method of claim 4, wherein said symptom of HD is a motor symptom of HD.

6. The method of claim 4, wherein said symptom of HD is a neurological or psychological symptom of HD.

7. The method of claim 5, wherein the motor symptom of HD is a symptom selected from the group of motor symptoms consisting of: involuntary jerking motions (spasms); involuntary writhing motions (chorea); muscular contractions or rigidity (dystonia); tremor; slowed or unusual eye movements; impaired muscle strength; impaired grasp; impaired gait (i.e., difficulty walking); impaired balance; impaired swallowing; impaired respiration; impaired posture; impaired ability to stand upright; impaired ability to maintain head position; and impaired speech,
where impairment is determined with comparison to baseline ability to perform the motor activity (e.g., before onset of HD symptoms, or upon initial diagnosis of HD symptoms),
wherein said administration of an effective amount of said heteroaryl ketone fused azadecalin GRM or of said octahydro fused azadecalin GRM is effective to treat said motor symptom of HD.

8. The method of claim 6, wherein the symptom of HD comprises epileptic seizures, and the treatment is effective to reduce the frequency of said epileptic seizures.

9. The method of claim 6, wherein the symptom of HD comprises epileptic seizures, and the treatment is effective to reduce the frequency or severity of said epileptic seizures.

10. The method of claim 6, wherein the symptom of HD is a neurological or psychological symptom selected from the group of neurological symptoms consisting of: amnesia; other memory loss; mental confusion; impaired speech; impaired ability to concentrate; impaired speed of comprehension; delirium; hallucinations; paranoia; depression; anxiety; apathy; and rapid or unprovoked changes in mood,
where impairment is determined with comparison to baseline ability or level of the neurological or psychological activity or symptom (e.g., before onset of HD symptoms, or upon initial diagnosis of HD symptoms),
wherein said administration of an effective amount of a heteroaryl ketone fused azadecalin GRM or of said octahydro fused azadecalin GRM is effective to treat said neurological or psychological symptom of HD.

11. The method of claim 1, wherein the GRM is a heteroaryl ketone fused azadecalin GRM, and said GRM comprises a chemical structure having the formula: 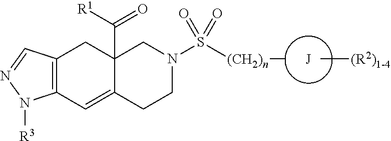
wherein
R¹ is a heteroaryl ring having from 5 to 6 ring members and from 1 to 4 heteroatoms each independently selected from the group consisting of N, O and S, optionally substituted with 1-4 groups each independently selected from R^{1a};
each R^{1a} is independently selected from the group consisting of hydrogen, C₁₋₆ alkyl, halogen, C₁₋₆ haloalkyl, C₁₋₆ alkoxy, C₁₋₆ haloalkoxy, CN, N-oxide, C₃₋₈ cycloalkyl, and C₃₋₈ heterocycloalkyl;
ring J is selected from the group consisting of a cycloalkyl ring, a heterocycloalkyl ring, an aryl ring and a heteroaryl ring, wherein the heterocycloalkyl and heteroaryl rings have from 5 to 6 ring members and from 1 to 4 heteroatoms each independently selected from the group consisting of N, O and S;
each R² is independently selected from the group consisting of hydrogen, C₁₋₆ alkyl, halogen, C_{1 6} haloalkyl, C_{1 6} alkoxy, C₁₋₆ haloalkoxy, C₁₋₆ alkyl-C₁₋₆ alkoxy, CN, OH, NR^{2a}R^{2b}, C(O)R^{2a}, C(O)OR^{2a}, C(O)NR^{2a}R^{2b}, SR^{2a}, S(O)R^{2a}, S(O)₂R^{2a}, C₃₋₈ cycloalkyl, and C₃₋₈ heterocycloalkyl, wherein the heterocycloalkyl groups are optionally substituted with 1-4 R^{2e} groups;
alternatively, two R² groups linked to the same carbon are combined to form an oxo group (═O);
alternatively, two R² groups are combined to form a heterocycloalkyl ring having from 5 to 6 ring members and from 1 to 3 heteroatoms each independently selected from the group consisting of N, O and S, wherein the heterocycloalkyl ring is optionally substituted with from 1 to 3 R^{2d} groups;
R^{2a} and R^{2b} are each independently selected from the group consisting of hydrogen and C₁₋₆ alkyl;
each R^{2c} is independently selected from the group consisting of hydrogen, halogen, hydroxy, C₁₋₆ alkoxy, C₁₋₆ haloalkoxy, CN, and NR^{2a}R^{2b};
each R^{2d} is independently selected from the group consisting of hydrogen and C₁₋₆ alkyl, or two R^{2d} groups attached to the same ring atom are combined to form (═O);
R³ is selected from the group consisting of phenyl and pyridyl, each optionally substituted with 1-4 R^{3a} groups;
each R^{3a} is independently selected from the group consisting of hydrogen, halogen, and C₁₋₆ haloalkyl; and
subscript n is an integer from 0 to 3;
or salts and isomers thereof.

12. The method of claim 11, wherein the heteroaryl ketone fused azadecalin GRM is dazucorilant (which is (R)-(1-(4-fluorophenyl)-6-((4-(trifluoromethyl)phenyl) sulfonyl)-4, 4a, 5,6,7,8-hexahydro-1-H-pyrazolo P,4-g]isoquinolin-4a-yl) (pyridin-2-yl)methanone), which has the following structure:

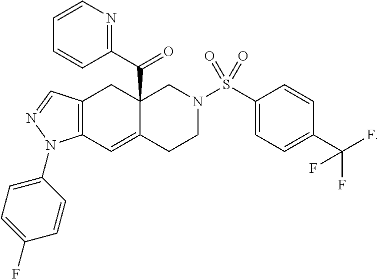 [SMILES: O=C(c1ccccn1)[C@]12Cc3cnn(-c4ccc(F)cc4)c3C=C1CCN(S(=O)(=O)c1ccc(C(F)(F)F)cc1)C2]

13. The method of claim 1, wherein the GRM is an octahydro fused azadecalin GRM, and said GRM comprises a chemical structure having the formula: 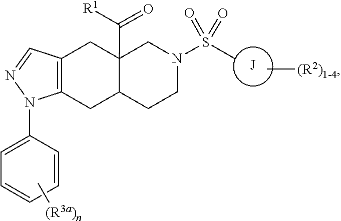
wherein
R¹ is a heteroaryl ring having from 5 to 6 ring members and from 1 to 4 heteroatoms each independently selected from the group consisting of N, O and S, optionally substituted with 1-4 groups each independently selected from R^{1a};
each R^{1a} is independently selected from the group consisting of hydrogen, C₁₋₆ alkyl, halogen, C₁₋₆ haloalkyl, C₁₋₆ alkoxy, C₁₋₆ haloalkoxy, N-oxide, and C₃₋₈ cycloalkyl; ring J is selected from the group consisting of an aryl ring and a heteroaryl ring having from 5 to 6 ring members and from 1 to 4 heteroatoms each independently selected from the group consisting of N, O and S;
each R² is independently selected from the group consisting of hydrogen, C₁₋₆ alkyl, halogen, C₁₋₆ haloalkyl, C₁₋₆ alkoxy, C₁₋₆ haloalkoxy, C₁₋₆ alkyl-C₁₋₆ alkoxy, CN, OH, NR^{2a}R^{2b}C(O)R^{2a}, C(O)OR^{2a}, C(O)NR^{2a}R^{2b}, SR^{2a}, S(O)R^{2a}, S(O)₂R^{2a}, C₃₋₈ cycloalkyl, and C₃₋₈ heterocycloalkyl having from 1 to 3 heteroatoms each independently selected from the group consisting of N, O and S;
alternatively, two R² groups on adjacent ring atoms are combined to form a heterocycloalkyl ring having from 5 to 6 ring members and from 1 to 3 heteroatoms each independently selected from the group consisting of N, O and S, wherein the heterocycloalkyl ring is optionally substituted with from 1 to 3 R^{2c} groups;
R^{2a}, R^{2b} and R^{2c} are each independently selected from the group consisting of hydrogen and C₁₋₆ alkyl;
each R^{3a} is independently halogen; and subscript n is an integer from 0 to 3,
or salts and isomers thereof.

14. The method of claim 13, wherein said octahydro fused azadecalin GRM is zavacorilant (which is ((4aR,8aS)-1-(4-fluorophenyl)-6-((2-isopropyl-2H-1,2,3-triazol-4-yl)sulfonyl)-4,4a,5,6,7,8,8a,9-octahydro-1H-pyrazolo[3,4-g]isoquinolin-4a-yl)(thiazol-4-yl)methanone), which has the following structure:

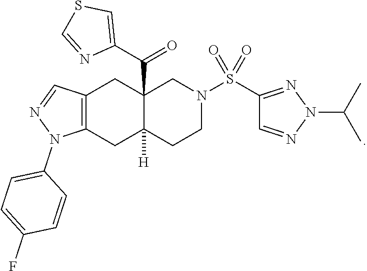 [SMILES: CC(C)n1ncc(S(=O)(=O)N2CC[C@H]3Cc4c(cnn4-c4ccc(F)cc4)C[C@]3(C(=O)c3cscn3)C2)n1]

15. A pharmaceutical composition for treating Huntington's Disease (HD) or a symptom thereof comprising a pharmaceutically acceptable excipient and a nonsteroidal glucocorticoid receptor modulator (GRM) compound comprising a heteroaryl ketone fused azadecalin structure having the formula: 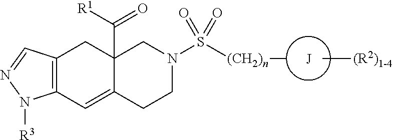
wherein
R¹ is a heteroaryl ring having from 5 to 6 ring members and from 1 to 4 heteroatoms each independently selected from the group consisting of N, O and S, optionally substituted with 1-4 groups each independently selected from R^{1a};
each R^{1a} is independently selected from the group consisting of hydrogen, C₁₋₆ alkyl, halogen, C₁₋₆ haloalkyl, C₁₋₆ alkoxy, C₁₋₆ haloalkoxy, CN, N-oxide, C₃₋₈ cycloalkyl, and C₃₋₈ heterocycloalkyl;
ring J is selected from the group consisting of a cycloalkyl ring, a heterocycloalkyl ring, an aryl ring and a heteroaryl ring, wherein the heterocycloalkyl and heteroaryl rings have from 5 to 6 ring members and from 1 to 4 heteroatoms each independently selected from the group consisting of N, O and S;
each R² is independently selected from the group consisting of hydrogen, C₁₋₆ alkyl, halogen, C_{1 6} haloalkyl, C_{1 6} alkoxy, C₁₋₆ haloalkoxy, C₁₋₆ alkyl-C₁₋₆ alkoxy, CN, OH, NR^{2a}R^{2b}C(O)R^{2a}, C(O)OR^{2a}, C(O)NR^{2a}R^{2b}, SR^{2a}, S(O)R^{2a}, S(O)₂R^{2a}, C₃₋₈ cycloalkyl, and C₃₋₈ heterocycloalkyl, wherein the heterocycloalkyl groups are optionally substituted with 1-4 R², groups;
alternatively, two R² groups linked to the same carbon are combined to form an oxo group (═O);
alternatively, two R² groups are combined to form a heterocycloalkyl ring having from 5 to 6 ring members and from 1 to 3 heteroatoms each independently selected from the group consisting of N, O and S, wherein the heterocycloalkyl ring is optionally substituted with from 1 to 3 R^{2d} groups;
R^{2a} and R^{2b} are each independently selected from the group consisting of hydrogen and C₁₋₆ alkyl;
each R^{2c} is independently selected from the group consisting of hydrogen, halogen, hydroxy, C₁₋₆ alkoxy, C₁₋₆ haloalkoxy, CN, and NR^{2a}R^{2b};
each R^{2d} is independently selected from the group consisting of hydrogen and C₁₋₆ alkyl, or two R^{2d} groups attached to the same ring atom are combined to form (═O);
R³ is selected from the group consisting of phenyl and pyridyl, each optionally substituted with 1-4 R^{3a} groups;
each R^{3a} is independently selected from the group consisting of hydrogen, halogen, and C₁₋₆ haloalkyl; and
subscript n is an integer from 0 to 3;
or salts and isomers thereof.

16. The pharmaceutical composition of claim 15, wherein the nonsteroidal GRM compound comprising a heteroaryl ketone fused azadecalin structure is dazucorilant (which is (R)-(1-(4-fluorophenyl)-6-((4-(trifluoromethyl)phenyl) sulfonyl)-4, 4a, 5,6,7,8-hexahydro-1-H-pyrazolo P,4-g]isoquinolin-4a-yl) (pyridin-2-yl)methanone), which has the following structure:

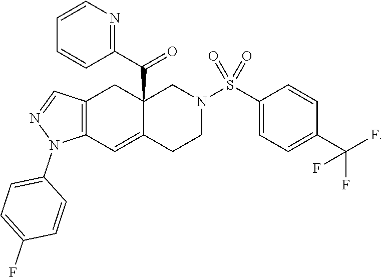 [SMILES: O=C(c1ccccn1)[C@]12Cc3cnn(-c4ccc(F)cc4)c3C=C1CCN(S(=O)(=O)c1ccc(C(F)(F)F)cc1)C2]

17. A pharmaceutical composition for treating Huntington's Disease (HD) or a symptom thereof comprising a pharmaceutically acceptable excipient and a nonsteroidal glucocorticoid receptor modulator (GRM) compound comprising an octahydro fused azadecalin structure having the formula: 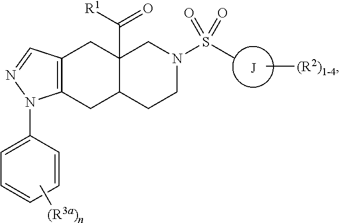
wherein
R¹ is a heteroaryl ring having from 5 to 6 ring members and from 1 to 4 heteroatoms each independently selected from the group consisting of N, O and S, optionally substituted with 1-4 groups each independently selected from R^{1a};
each R^{1a} is independently selected from the group consisting of hydrogen, C₁₋₆ alkyl, halogen, C₁₋₆ haloalkyl, C₁₋₆ alkoxy, C₁₋₆ haloalkoxy, N-oxide, and C₃₋₈ cycloalkyl; ring J is selected from the group consisting of an aryl ring and a heteroaryl ring having from 5 to 6 ring members and from 1 to 4 heteroatoms each independently selected from the group consisting of N, O and S;
each R² is independently selected from the group consisting of hydrogen, C₁₋₆ alkyl, halogen, C₁₋₆ haloalkyl, C₁₋₆ alkoxy, C₁₋₆ haloalkoxy, C₁₋₆ alkyl-C₁₋₆ alkoxy, CN, OH, NR^{2a}R^{2b}C(O)R^{2a}, C(O)OR^{2a}, C(O)NR^{2a}R^{2b}, SR^{2a}, S(O)R^{2a}, S(O)₂R^{2a}, C₃₋₈ cycloalkyl, and C₃₋₈ heterocycloalkyl having from 1 to 3 heteroatoms each independently selected from the group consisting of N, O and S;
alternatively, two R² groups on adjacent ring atoms are combined to form a heterocycloalkyl ring having from 5 to 6 ring members and from 1 to 3 heteroatoms each independently selected from the group consisting of N, O and S, wherein the heterocycloalkyl ring is optionally substituted with from 1 to 3 R^{2c} groups;
R^{2a}, R^{2b} and R^{2c} are each independently selected from the group consisting of hydrogen and C₁₋₆ alkyl;
each R^{3a} is independently halogen; and subscript n is an integer from 0 to 3,
or salts and isomers thereof.

18. The pharmaceutical composition of claim 17, wherein the nonsteroidal GRM compound comprising an octahydro fused azadecalin GRM is zavacorilant (which is ((4aR,8aS)-1-(4-fluorophenyl)-6-((2-isopropyl-2H-1,2,3-triazol-4-yl)sulfonyl)-4,4a,5, 6,7,8,8a,9-octahydro-1H-pyrazolo[3,4-g]isoquinolin-4a-yl)(thiazol-4-yl)methanone), which has the following structure:

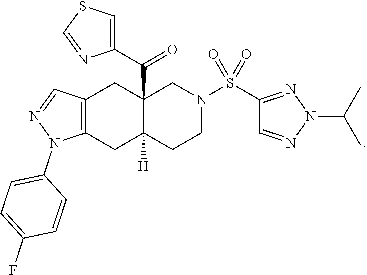 [SMILES: CC(C)n1ncc(S(=O)(=O)N2CC[C@H]3Cc4c(cnn4-c4ccc(F)cc4)C[C@]3(C(=O)c3cscn3)C2)n1]

19. The pharmaceutical composition of claim 15, for treating a symptom of HD, wherein said symptom of HD is a motor symptom of HD.

20. The pharmaceutical composition of claim 15, for treating a symptom of HD, wherein said symptom of HD is a neurological or psychological symptom of HD.

21. The pharmaceutical composition of claim 17, for treating a symptom of HD, wherein said symptom of HD is a motor symptom of HD.

22. The pharmaceutical composition of claim 17, for treating a symptom of HD, wherein said symptom of HD is a neurological or psychological symptom of HD.

23-35. (canceled)
